(19) United States
(12) Patent Application Publication
(10) Pub. No.: US 2009/0185981 A1
(43) Pub. Date: Jul. 23, 2009

(54) METHODS AND APPARATUS FOR DYNAMICALLY ALLOCATING BANDWIDTH TO SPECTRAL, TEMPORAL, AND SPATIAL DIMENSIONS DURING A MAGNETIC RESONANCE IMAGING PROCEDURE

(76) Inventors: Gregory Karczmar, Crete, IL (US); Milica Medved, Chicago, IL (US); Gillian Newstead, Chicago, IL (US)

Correspondence Address: MICHAEL BEST & FRIEDRICH LLP, 100 E WISCONSIN AVENUE, Suite 3300, MILWAUKEE, WI, 53202, US

(21) Appl. No.: 12/294,201

(22) PCT Filed: Mar. 23, 2007
(86) PCT No.: PCT/US07/64836

§ 371 Date: Feb. 5, 2009

Related U.S. Application Data
(60) Provisional application No. 60/785,868, filed on Mar. 24, 2006.

(51) Int. Cl. A61B 5/055; G01R 33/48

(52) U.S. Cl. 424/9.3; 324/309

(57) ABSTRACT

A system and method of dynamically allocating signal acquisition bandwidth in magnetic resonance imaging systems. The use of high spatial and high spectral resolution in MRI imaging can improve the clinical usefulness of the images. However, during uptake and washout of contrast agents, the use of high spatial and high spectral resolution results in important information being missed. Dynamic allocation of MRI signal acquisition bandwidth allows the use of high temporal resolution during contrast agent uptake and washout and high spatial and spectral resolution during periods of slower morphology resulting in images containing additional data than in conventional MRI protocols.

### RELATED APPLICATIONS

This application claims the benefit of U.S. Provisional Patent Application No. 60/785,868, filed on Mar. 24, 2006, entitled METHODS AND APPARATUS FOR DYNAMICALLY ALLOCATING BANDWIDTH TO SPECTRAL, TEMPORAL, AND SPATIAL DIMENSIONS DURING A MAGNETIC RESONANCE IMAGING PROCEDURE, the content of which is incorporated herein by reference.

### STATEMENT REGARDING FEDERALLY SPONSORED RESEARCH OR DEVELOPMENT

This invention was made with government support under R21 CA104774 awarded by the NIH. The government has certain rights in the invention.

### BACKGROUND

Early detection and accurate characterization of many medical conditions, such as breast cancer, are critical to the successful clinical management of the condition. Intervention at an early stage can greatly reduce morbidity and mortality. Magnetic resonance imaging (MRI) has proven to be an effective tool in this early detection and characterization. However, it is critical that the number of false positives (e.g., lesions incorrectly identified as cancer) be minimized. In the case of detection and characterization of breast cancer for example, large numbers of women can be subjected to the stress, discomfort, and cost of unneeded biopsies without high specificity. Improvements in specificity (i.e., reductions in the false positive rate) are critical if highly sensitive MRI methods are to be used routinely for a second stage screening procedure or even for routine screening of high-risk medical conditions.

Contrast-enhanced MRI significantly increases the ability of physicians to detect conditions such as breast cancer. However, specificity, to date, has not been satisfactory. In fact, the very high sensitivity of MRI places great demands on specificity in order to avoid large numbers of false positives. Despite the efforts of many researchers to improve dynamic, contrast-enhanced MRI, the specificity remains below an acceptable level. The very high sensitivity of MRI can magnify the unacceptably low specificity. In addition, sensitivity is inadequate for early detection of some conditions (e.g., early forms of breast cancer, such as ductal carcinoma in situ).

Although MRI has the potential to improve sensitivity and accuracy of detection of medical conditions (such as breast cancer), to date, it has not proven to be sufficiently accurate in many applications to be used routinely by clinicians.

### SUMMARY

Previous work has demonstrated that high spectral and spatial resolution MRI improves image contrast and anatomic detail. This spectral/spatial imaging approach has not previously been applied to dynamic imaging (e.g., for imaging of contrast media uptake) with high temporal resolution due to the time required for spectral/spatial imaging. With improvements in MRI technology, the technical barriers to dynamic spectral and spatial resolution imaging with high temporal resolution no longer exist. The use of high temporal resolution and moderate spatial and spectral resolution after, for example, contrast media injection, and higher spatial and spectral resolution with lower temporal resolution during contrast media washout provides significant benefits and physiologic and morphologic information. This method of dynamically allocating bandwidth during an imaging procedure can significantly improve analysis of an image, such as by providing high temporal resolution combined with modest spectral resolution during times when image contrast is changing rapidly (e.g., immediately following contrast media injection) with increasing spectral and spatial resolution during times when image contrast is changing more slowly. This allows accurate separation of fat and water signals and measurement of effects of contrast agents on T₂*, T₁, and resonance frequency. This method can also optimize the functional and morphological information obtained, and can increase sensitivity to the angiogenic, invasive, and morphologic properties of the imaged matter (e.g., breast lesions, in some applications).

The present invention relates to MRI systems and methods, and specifically to imaging processes wherein the desired imaging method varies over the imaging session. More specifically, the invention relates to dynamically allocating bandwidth to the spatial, spectral, and temporal dimensions.

In some embodiments, the invention provides a method of generating magnetic resonance images of a patient. The method includes the acts of allocating a bandwidth for temporal resolution, spatial resolution, and spectral resolution, acquiring images of the patient, and semi-automatically modifying the bandwidth allocation of at least one of the temporal resolution, spatial resolution, and spectral resolution at the expense of at least one of the other two resolutions.

In some embodiments, the invention provides a magnetic resonance imaging system comprising a housing and a computer program. The housing includes means for acquiring images of a patient. The computer program includes a setup module operable to allocate a bandwidth for temporal resolution, spatial resolution, and spectral resolution, and a scanning module operable to semi-automatically modify the bandwidth allocation of at least one of the temporal resolution, the spatial resolution, and the spectral resolution at the expense of at least one of the other two resolutions.

In other embodiments, the invention provides a method of generating a set of magnetic resonance images of a patient by injecting the patient with a contrast agent, and implementing a first imaging protocol which allocates the MRI signal acquisition bandwidth to high temporal resolution and high spatial resolution. Following a predetermined time period in which the contrast agent has entered the region of interest, a second imaging protocol is automatically implemented. The second imaging protocol allocates the MRI signal acquisition bandwidth to high spatial resolution and high spectral resolution.

In still other embodiments, the present invention provides a method of identifying diagnostic markers for magnetic resonance imaging. A protocol is developed that includes a standard clinical procedure, and is enhanced to include a dynamically allocated MRI bandwidth imaging protocol. Data from the images obtained by the dynamically allocated MRI bandwidth protocol can be analyzed and compared with data obtained from the standard clinical procedure. Data that is determined to be relevant can be designated as an effective diagnostic marker.

Other aspects of the invention will become apparent by consideration of the detailed description and accompanying drawings.

### BRIEF DESCRIPTION OF THE DRAWINGS

FIG. 1 is a block diagram of an embodiment of a MRI system.

FIGS. 2A-2F illustrate a comparison of images obtained by conventional MRI versus high spatial and spectral resolution MRI methods.

FIGS. 3A and 3B illustrate a comparison of a HiSS MRI image with an image of the difference between the image of FIG. 3A and the same image taken 3 minutes after injection of a contrast agent.

FIGS. 4A and 4B graphically illustrate the spectral waterline before and after injection of contrast agent for two separate pixels in a tumor shown in FIGS. 3A and 3B.

FIG. 5 graphically illustrates an up-take and washout rate of a contrast agent for several patients with different types of tumors.

FIG. 6 is a representation of an exemplary pulse sequence of an echo-planar spectroscopic imaging MRI for obtaining lines in k-space in parallel.

FIG. 7 illustrates a sequence for a protocol using dynamically allocated bandwidth MRI.

### DETAILED DESCRIPTION

Before any embodiments of the invention are explained in detail, it is to be understood that the invention is not limited in its application to the details of construction and the arrangement of components set forth in the following description or illustrated in the following drawings. The invention is capable of other embodiments and of being practiced or of being carried out in various ways. Also, it is to be understood that the phraseology and terminology used herein is for the purpose of description and should not be regarded as limiting. The use of “including,” “comprising,” or “having” and variations thereof herein is meant to encompass the items listed thereafter and equivalents thereof as well as additional items.

In addition, it should be understood that embodiments of the invention include hardware, software, and electronic components or modules that, for purposes of discussion, may be illustrated and described as if the majority of the components were implemented solely in hardware. However, one of ordinary skill in the art, and based on a reading of this detailed description, would recognize that, in at least one embodiment, the electronic based aspects of the invention may be implemented in software. As such, it should be noted that a plurality of hardware and software based devices, as well as a plurality of different structural components, may be utilized to implement the invention. Furthermore, and as described in subsequent paragraphs, the specific mechanical configurations illustrated in the drawings are intended to exemplify embodiments of the invention. Other alternative mechanical configurations are possible.

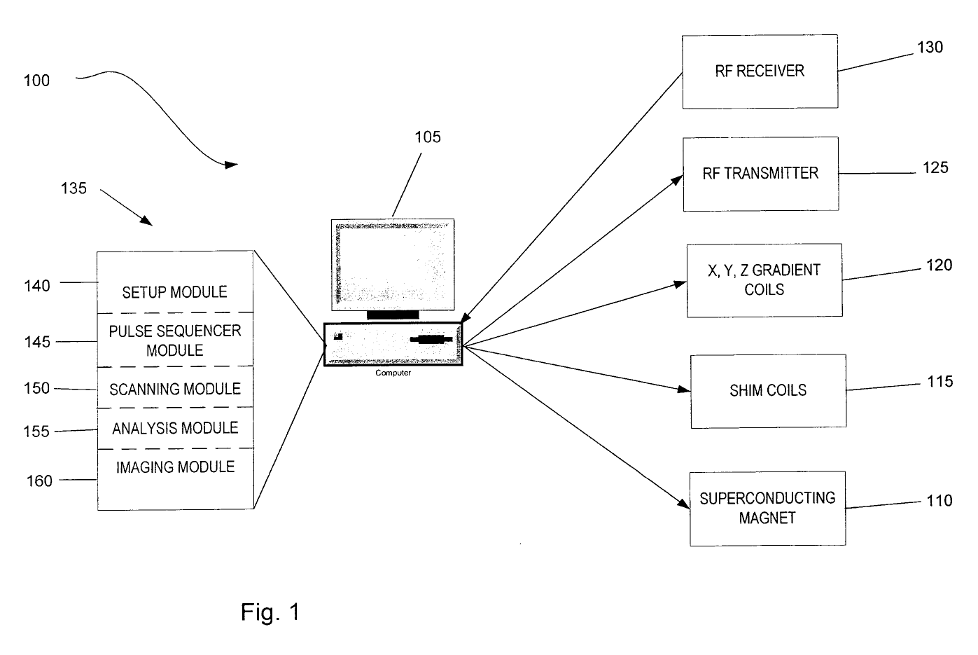

FIG. 1 illustrates a MRI system 100 according to one embodiment of the present invention. The MRI system 100 includes a computer 105, a superconducting magnet 110, a set of shim coils 115, a set of gradient coils 120, a radio frequency (“RF”) transmitter coil 125, and a RF receiver 130.

The MR imaging system 100 functions to generate images by operating on MR detectable nuclei (e.g., a hydrogen proton) with a combination of static and radio frequency magnetic fields applied through the superconducting magnet 110 and the set of gradient coils 120, and shim coils 115. Radiofrequency energy applied by the RF transmitter coil 125, at the Larmor frequency, perturbs the nuclear magnetic moments away from their equilibrium state, and this results in the release of energy during free induction decay (“FID”). This release of energy can be detected by the RF receiver 130 and can be provided to the computer 105.

The computer 105 can include an operating system for running various software programs and/or communication applications. In particular, the computer 105 can include a software program or programs 135 that facilitate communication between the computer 105 and the superconducting magnet 110, shim coils 115, gradient coils 120, and radio frequency (“RF”) transmitter coil 125, and can provide an operator interface to the MRI system 100. The software program 135 can include a setup module 140, a pulse sequencer module 145, a scanning module 150, an analysis module 155, and a display module 160. The computer 105 can include suitable input/output devices adapted to be accessed by medical personnel or technicians. The computer 105 can include typical hardware such as a processor, I/O interfaces, and storage devices or memory. The computer 105 can also include input devices such as a keyboard and a mouse, and/or output devices such as a monitor. In addition, the computer 105 can include peripherals, such as a printer and a scanner.

In some embodiments, the setup module 140 can enable an operator to create an imaging protocol for an imaging session. The imaging protocol can at least partially define where to obtain images from, what orientation to image at, and which imaging method to use. In some embodiments, the setup module 140 can enable multiple protocols to be sequenced together based on triggering events (e.g., time, a detected condition, a user command or act, and the like).

Based on the protocol or protocols entered into the setup module 140, the pulse sequencer module 145 can create a series of pulse sequences for the gradient coils 120 and the RF transmitter coil 125 for each protocol. Once a patient is in position and the superconducting magnet 110 is up to full power, the scanning module 150 can execute the pulse sequences created by the pulse sequence module 145 and can receive the data from the RF receiver 130 reflecting the energy released during the FID.

The data received from the RF receiver 130 can be stored in the computer system 105. The analysis module 155 can manipulate the data from the RF receiver 130 (whether stored on the computer system 105 or otherwise) to improve image quality and/or to accentuate features in the images. The display module 160 can display an image of the data received from the RF receiver 130 for an operator either in a raw form or following manipulation by the analysis module 155.

The use of high spectral and spatial resolution (“HiSS”) in the acquisition of MRI data produces images that include, for example, data on water peak height, line width, resonance frequency, and other features of the water and fat line shapes in voxels to a much greater degree than high spatial resolution imaging alone. HiSS MRI data is acquired at a spatial resolution equivalent to that of conventional anatomic imaging or higher, and at a high spectral resolution (e.g., about 2-15 Hz). Some of the advantages of HiSS imaging include:

images with improved anatomic detail,

images with improved fat/water separation,

images corrected for B₀ inhomogeneity,

increased sensitivity to MRI contrast agents,

diagnostically valuable images obtained prior to contrast agent injection, and

images synthesized from various Fourier components of the water resonance, providing unique functional and anatomic information.

In some embodiments, HiSS images can also provide the advantage of effective fat saturation. In this manner, the ability to detect some medical conditions (e.g., breast lesions) without the need for contrast media injection can be significantly increased.

Some HiSS images can also detect water protons in various subvoxelar and microscopic environments that are functionally and/or anatomically distinct. These distinctions can cause the water protons to respond to contrast agents in different ways. The imaging methods that incorporate a higher degree of spectral resolution (e.g., HiSS) than conventional anatomic imaging can have increased sensitivity to these effects. This increased sensitivity can provide a clinician with valuable information not available with imaging methods that do not incorporate a high degree of spectral resolution.

For example, a change in resonance frequency, T₁ or T₂*, of a small shoulder of a water resonance, may be associated with a subvoxelar region of dense vasculature that can be easily detected with HiSS MRI but may be impossible to detect with conventional MRI.

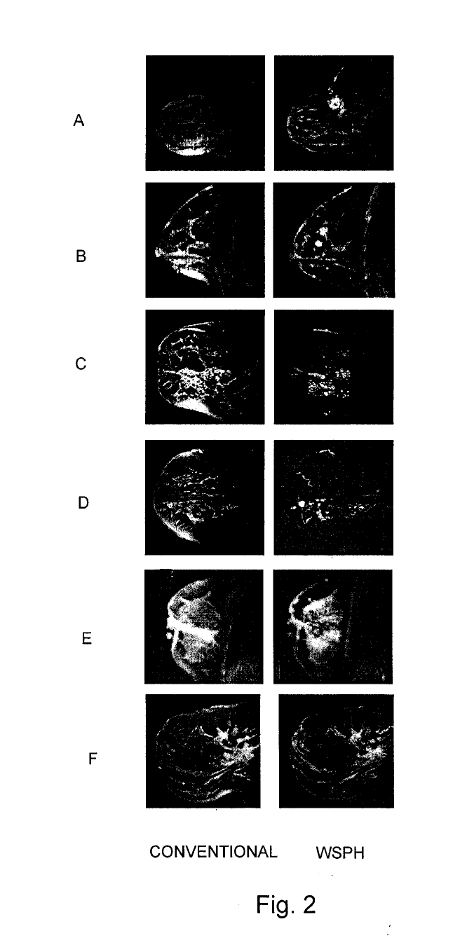

FIGS. 2A-F illustrate several examples of non-contrast enhanced HiSS images (right column) of suspicious breast lesions compared with conventional T₁-weighted fat-saturated post contrast injection images (left column). A HiSS water signal peak height (“WSPH”) image can have intensity proportional to the peak height of the water resonance in each voxel. A conventional fat saturation image is non-uniform, and even where saturation is effective, residual fat signal due to inhomogeneous broadening of the fat resonance exists. As shown in FIGS. 2A-F, separation of fat and water signals is improved in the HiSS images. Anatomy can be clearer in HiSS images than in conventional T₁-weighted fat-saturated post contrast injection images. In particular, edges and duct configurations can be more clearly defined. Lesions appear clearly in HiSS images even in the absence of contrast media. In addition, morphology not shown in a conventional image is often shown in a HiSS image.

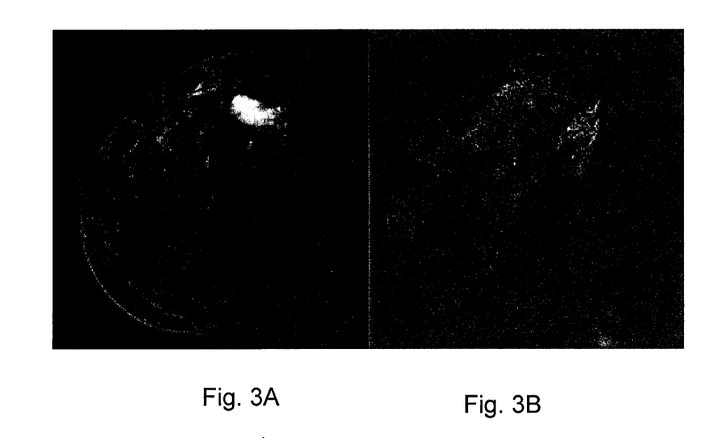

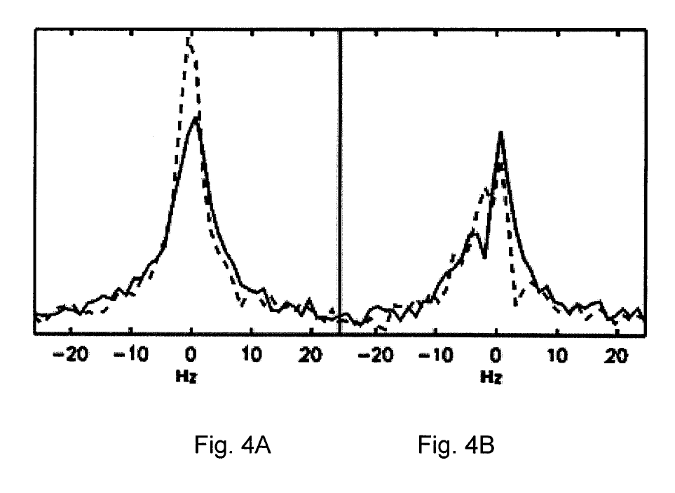

FIGS. 3A and 3B provide examples of some of the important and novel information available in contrast enhanced HiSS imaging. For example, analysis of changes in the water line-shape in each small image voxel, due to contrast agents, may show spectrally inhomogeneous changes in the water signal in many voxels. This novel contrast may be clinically useful. For example, FIG. 3A shows a HiSS water peak height image and FIG. 3B shows a water peak height difference image obtained from a HiSS image acquired 3 minutes after a contrast agent injection. Even though the post-contrast image was acquired relatively long after the contrast agent injection, the difference image shows signal (both T₂*-weighted and T₁-weighted) changes with great detail. FIGS. 4A and 4B graphically illustrate spectra data for two individual voxels in the tumor shown in FIGS. 3A and 3B. The spectra graph before (dashed line) and after (solid line) contrast agent injection demonstrates resonance frequency shifts and changes in line shape due to the contrast agent. Many of the spectral changes detected with HiSS following contrast agent injection would be difficult to detect in conventional images. For example, frequency, amplitude, or T₂* changes in small “shoulders” of the water resonance would have only a small effect, if any, on the intensity of conventional T₁-weighted images. However, these features can be clearly seen in HiSS images.

High spectral and spatial resolution can improve functional imaging due to the increased sensitivity to the effects of endogenous contrast agents (e.g., deoxyhemoglobin) and injected contrast agents. This is especially true when the effects of the contrast agents are spectrally inhomogeneous (i.e., the contrast agents have different effects on different components of an inhomogeneously broadened water resonance in each voxel).

Rapid acquisition of echo-planar spectroscopic images (“EPSI”) at very high spectral and spatial resolution and with minimal eddy current distortion is possible because of advances in MRI hardware and software. As a result, details of the water and fat line shapes in each small image pixel can be resolved within reasonable acquisition times. In addition, imaging methods based on analysis of a train of gradient echoes can reduce the effects of B₀ inhomogeneity and increase T₂* contrast, providing greatly improved image quality over previous methods.

Parallel imaging has also been implemented on MRI scanners (e.g., SENSE™ technology from Philips Medical Systems). Parallel imaging software can be adapted for use with spectral/spatial datasets, and to follow standard protocols. Protocols can be improved by increasing acceleration factors and using accurate phase information. This phase information is inherent in the spectral information of each voxel detected by a coil element.

In addition, optimized shimming can provide excellent B₀ homogeneity across the scan area. Bₒ homogeneity is typically a critical element for HiSS imaging.

Spectral/spatial datasets can be analyzed to produce parametric images with intensity proportional to water/fat signal peak height, resonance frequency, and/or T₂*.

Errors in timing and eddy currents can lead to k-space sampling errors, particularly in high-resolution EPSI data. These errors do not vary markedly from patient to patient. Therefore, sampling errors can be measured using a simple spherical phantom placed at various positions in a radio frequency (“RF”) coil. The water signal from the phantom can be shimmed to a fraction of a Hertz so that there is no resonance offset effects and minimal line broadening effects in spectral/spatial data acquired from the phantom. Since the true k-space representation of the phantom can be accurately calculated, deviations from the k-space representation at each sample point (i.e., gradient echo) along the FID due to timing errors and/or eddy currents can be determined and these deviations can be corrected in the data. The parameters used for shimming the EPSI of the phantom can also be used for a patient.

HiSS datasets can span a large spatial/spectral parameter space. Poor signal to noise ratio (“SNR”) can result in a significant portion of the parameter space providing information that is not useful. A variety of 3-D filters can optimize the SNR while preserving the spectral and spatial information. For example, the standard deviation for the Gaussian that multiplies each readout gradient echo along the FID can have an exponential dependence on the echo number and a Gaussian dependence on k_{y}, and can be used to attenuate noise at very high kₓ and k_{y} values, and for late echoes.

Contrast agents have been used in MRI for a number of years and contribute to or instigate changes in the human body when injected. These agents can cause the brightness of various parts of a body (where the agent is residing) to increase in MRI images. Most contrast agents are extracellular, and reside for a relatively short time in the vascular system. However, some contrast agents are intracellular, and can reside for a relatively longer time in the vascular system. High-resolution spectroscopic imaging can show the different effects contrast agents have on intracellular and extracellular environments.

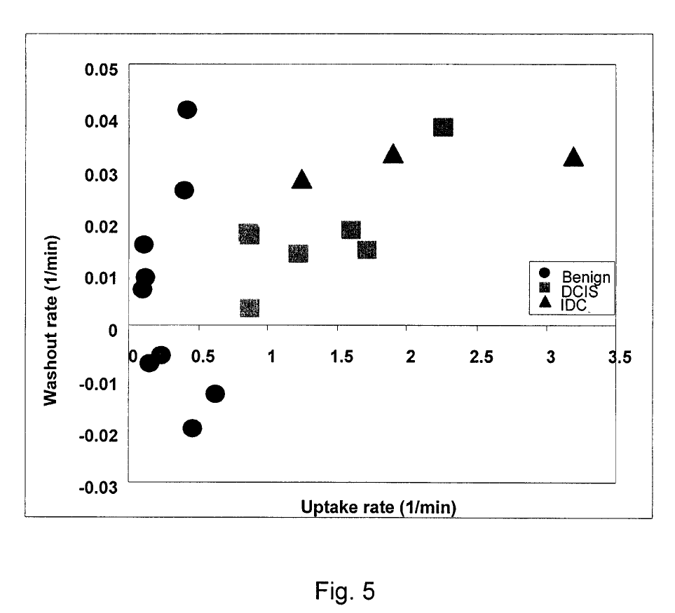

While HiSS imaging provides information not found in conventional MRI, more detailed analysis of contrast media uptake and washout rates may improve diagnostic accuracy even further. For example, FIG. 5 shows a plot of contrast uptake rate vs. washout rate for several patients with breast lesions. The plot shows a separation between benign and malignant breast lesions. This imaging of contrast media kinetics may provide data that is relevant to the diagnosis of cancer.

Contrast agents injected intravenously have been shown to have spectrally inhomogeneous effects in small image voxels in the human breast and in rodent tumors. For example, HiSS images of rodent tumors have shown that the effects of carbogen inhalation on the water resonance in small voxels are spectrally inhomogeneous.

Contrast media uptake and washout rates can be relatively rapid. Therefore, to detect changes in morphology that may occur during contrast agent uptake and washout, it is advantageous to use high temporal resolution to obtain images during contrast agent uptake and washout.

MRI signal acquisition bandwidth is finite and limited. In order to increase one type of signal resolution (e.g., temporal), such as for reasons described above, it is often necessary to reduce one or more of the other signal resolutions (e.g., spatial and/or spectral). Advances in technology have achieved better resolutions. These increases, however, are limited by certain factors (e.g., T₁, T₂*, FID) that do not change. This bandwidth limitation restricts the amount and type of images that can be acquired by the MRI system 10. Dynamic allocation of MRI signal acquisition bandwidth can allow the MRI system 10 to acquire images utilizing the available bandwidth in a more effective manner. The allocation of MRI signal acquisition bandwidth can vary dynamically based on events occurring in a region of interest (“ROI”), providing images in which the most important element(s) are emphasized. Broadly speaking, the ROI may include the entire body, but is generally smaller than the entire body and can be defined by a two-dimensional area and/or a three-dimensional volume.

Modification of the allocation of MRI signal acquisition bandwidth can occur automatically, semi-automatically, or manually one or more times during a MRI imaging protocol. An operation of the system performed semi-automatically can be partially performed by the system 10 and partially performed by the user of the system 10. For example, semi-automatic processes include user interaction with the system 10 to initiate and/or confirm processes to be performed by the system 10. The system 10 operates to perform various protocols that may request input or confirmation from the user to continue the protocol(s) or process.

For example, during the uptake of a contrast agent bolus, when changes are occurring rapidly, relatively high temporal resolution can be used to obtain images that reflect the changes occurring. Therefore, it may be necessary to sacrifice spatial and/or spectral resolution to achieve the temporal resolution necessary for imaging contrast media uptake and washout.

The conventional approach to achieving this high temporal resolution has been to acquire dynamic contrast enhanced MRI (“DCEMRI”) data with very rapid spoiled gradient echo imaging. This approach provides a high temporal resolution and an acceptable signal-to-noise ratio, but does not include any spectral resolution. Also, this allocation of MRI signal acquisition bandwidth to temporal resolution may not be the most effective imaging means at other times during the imaging process. Dynamic allocation of MRI signal acquisition bandwidth allows the allocation of bandwidth to be modified throughout an imaging procedure. For example, the bandwidth allocation for high temporal resolution necessary during contrast agent uptake can be modified to accommodate a bandwidth allocation for high spatial and spectral resolution during later imaging times when changes are occurring at a slower pace.

During rapid DCEMRI, fat saturation is often performed to eliminate motion artifacts or small changes in the T₁ or T₂* of the fat in difference images. Fat saturation, however, has some disadvantages including:

fat saturation does not work well in some parts of the body because of macroscopic Bₒ field gradients (improved shimming and saturation pulses have not totally resolved this problem),

potential information in the fat resonance is removed by fat saturation (however, this information can be shown in HiSS images),

fat saturation may cause some magnetization transfer leading to a loss of water signal,

water resonance may be affected during the first pass of the contrast media bolus when the water resonance may become quite broad and its frequency may shift significantly due to a large intravascular concentration of contrast agent, accurate fat saturation requires a long saturation pulse during which a signal cannot be acquired, and

power deposition due to efficient fat saturation can become significant at higher magnetic fields.

As an alternative to fat saturation in DCEMRI, data is often acquired after a TE that is set so that fat and water magnetization are in-phase at the beginning of data acquisition. This reduces artifacts due to changes in destructive interference between water and fat signals. However, this approach is subject to error, and results in a loss of data during the initial part of the proton FID when the signal is largest and contains valuable information. In addition, it is important to maximize the amount of information about the water signal acquired during the initial uptake period when sensitivity to tumor blood flow is greatest.

HiSS imaging can be used as an alternative to fat saturation. HiSS imaging places greater demands on scanner performance than fat saturation; however, it has advantages that make it a viable alternative to fat saturation. One advantage of HiSS imaging is improved image contrast and anatomic detail. In order to incorporate high levels of spectral resolution in an image, it is necessary to use relatively low temporal resolution. Thus, HiSS imaging has not been applied to imaging of contrast media uptake with high temporal resolution because of the long time required for spectral/spatial imaging. However, improvements in MRI technology enable dynamic allocation of MRI bandwidth to spectral, spatial, and temporal resolution.

By using dynamic allocation of MRI bandwidth, the high temporal resolution necessary for imaging during contrast agent uptake can be combined with HiSS imaging following the contrast agent uptake.

Water and fat resonances can be phased to obtain a “pure absorption component.” This phasing can increase sensitivity to the detailed shape of the water and fat resonances and to the effects of contrast agents, and can increase SNR. However, even small errors in phasing can cause artifacts. Therefore, very robust phasing programs that work for water and fat resonances are used. These phasing programs can work with high-resolution spectral/spatial datasets.

In some embodiments, synthesis of images from HiSS datasets requires identification of the fat and water resonances in each image voxel. Non-uniformity of the magnetic field can cause variations in the resonance frequencies of water and fat which are locally small but can be globally large. To reduce the effects of non-uniform magnetic fields, the water and fat signals are identified based on a resonance offset relative to already identified neighboring voxels.

First, the largest spectral peak can be identified in all image voxels. Beginning with the voxel with the highest intensity spectral peak, a region growing procedure identifies water and fat signals based on a resonance offset relative to already-identified neighboring voxels. The first instance in which the offset is larger than a few spectral bins (arising from small local gradients and/or physiological noise) identifies an initial spectral peak based on the known relative positions of the water and fat resonances. The voxels are selected in order of decreasing signal intensity from a neighborhood of already identified voxels, guaranteeing that frequency map information is derived from voxels with the highest SNR. Then, the water resonance frequency is calculated in each pixel that is predominantly fat using the appropriate chemical-shift offset, and a fat peak is similarly identified in each pixel that is predominantly water. Fold-back effects arising from the periodic behavior of the Fast Fourier Transform (“FFT”) are accounted for during the process. Images can then be calculated with intensity proportional to water resonance peak height, fat resonance peak height, T₂*, and resonance frequency.

Embodiments of dynamic spectral/spatial imaging can use parallel sampling of multiple k-space lines to reduce scan times. High spectral and spatial resolution images can also be acquired using EPSI. Following slice selective excitation and a phase encoding gradient pulse, ‘readout’ gradient echoes can be acquired using trapezoidal gradient pulses with alternating polarity. For the purposes of the following discussion, the phase encoding gradients sample the ‘k_{y}’ direction and the readout gradients sample lines along the ‘k_{X}’ direction. The oscillating readout gradient can produce a ‘train’ of gradient echoes that modulates the proton FID. Each gradient echo samples a line along kₓ, at a different TE. A “crusher” gradient is applied at the end of the echo train to eliminate residual transverse magnetization. This approach can yield excellent images and spectra without eddy current distortion

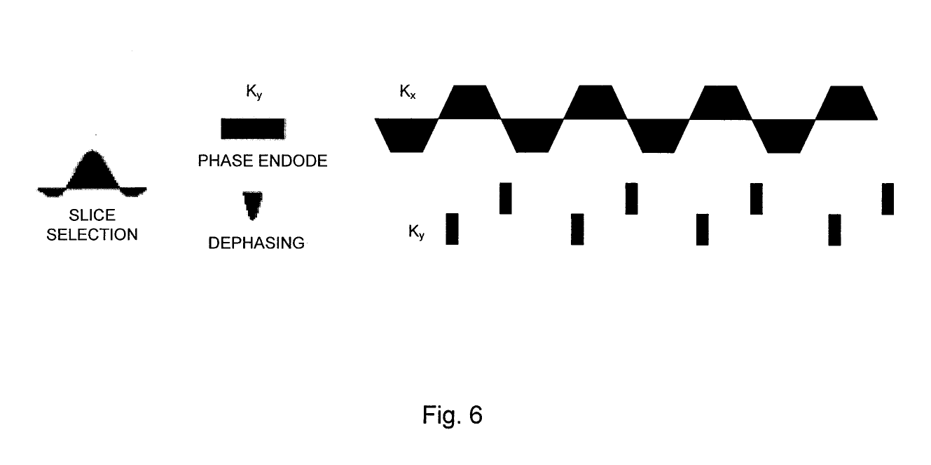

In some embodiments of dynamically allocated MRI, sample images are made with reduced spectral resolution during a period of time during an imaging session, such as during the initial uptake of contrast media. Two or more lines of k-space can be sampled in parallel (i.e., two or more values of k_{y} for each line along kₓ). Sampling of two or more k-space lines in parallel can also reduce the signal-to-noise ratio (“SNR”). The data, however, is not SNR-limited. Improvements in SNR due to improved data filtering and processing and improved RF coils can offset the loss in SNR due to high bandwidth sampling of multiple lines of k-space. In some embodiments, eight echoes for each line along kₓ are acquired to provide modest spectral resolution, but high temporal resolution. FIG. 6 shows an EPSI sequence with two k-space lines sampled in parallel. In some embodiments, 4 or 8 lines may be sampled and increase scan speed while maintaining an acceptable SNR. Phase encoding ‘blips’ of alternating polarity can be applied between the readout gradient echoes, to allow sampling of lines along kₓ at two different values of k_{y}. Each gradient echo can sample 256 points with a data acquisition bandwidth of about 250 kHz, and with 256 phase encoding steps. A gradient strength of 3.3 G/cm with rise times of approximately 160 microseconds can be used. This can allow sub-millimeter resolution with gradient echo durations of 1.5 msec or less (including time for gradient switching). The FID can be sampled for about 24 msec, with a spectral resolution of about 42 Hz by acquiring sixteen echoes.

Acceleration factors of at least 2 for parallel imaging, of 2 for sampling at least 2-lines of k_{y} in parallel, and of 1.5 for partial k-space sampling, enable a slice to be imaged in approximately 1 second. Eight slices can be imaged through a lesion and surrounding tissue with a time resolution of about 8 seconds or less. More efficient sampling of the FID may improve the time resolution even further.

In some embodiments, sampling with higher spectral and/or spatial resolution can take place. For example, following contrast media uptake, during the relatively slower phase of contrast media distribution, sampling with higher spectral and spatial resolution can take place. In such embodiments, this change can occur at approximately three minutes after contrast media injection. At this point, the contrast media concentration may be changing slowly, and bandwidth can be dynamically allocated to spectral and spatial resolution at the expense of temporal resolution. In some embodiments, a matrix size of 256 by 256 is sampled with spectral resolution of 5 Hz. Parallel imaging, reduced k-space sampling, and sampling 2-4 lines of k-space in parallel can result in an acquisition time of about 10 seconds or less per slice. Multiple slices can be imaged during the relatively slow phase of washout. Between 20 and 30 slices can be imaged with time resolution of about 4 minutes. In some embodiments, further increases in speed can be achieved, for example, by sampling eight or more lines in k-space in parallel.

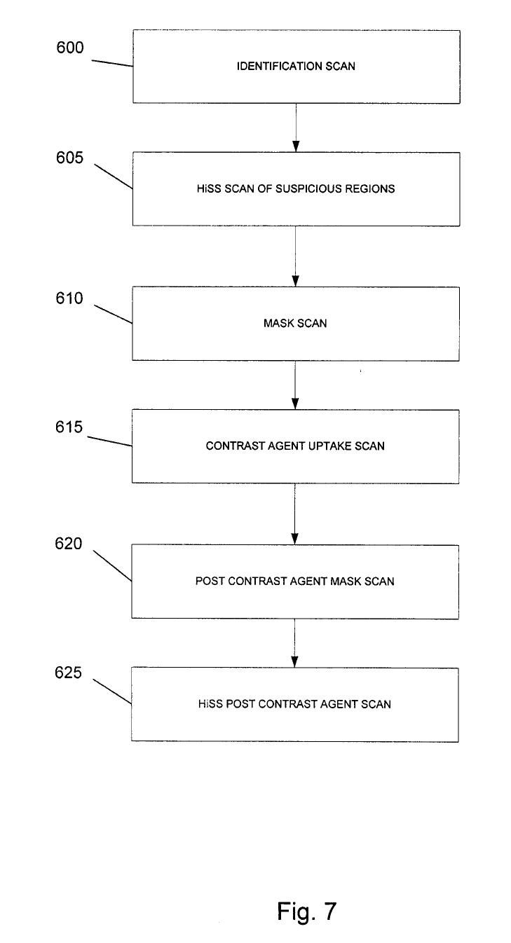

FIG. 7 shows a sequence for a sample imaging protocol using dynamic allocation, according to one embodiment of the method of the present invention, for a woman who presents with a suspicious breast lesion. An identification scan (block 600) is performed to look for abnormalities. The scan is a bilateral HiSS scan with moderate spectral and spatial resolution. Properties of the identification scan can include, for example:
Spectral: 25 Hz
Spatial: 1.0 mm×1.0 mm×3.0 mm voxel
# of Slices: 160 saggital
Time: about 8 minutes with a SENSE acceleration factor of 3

From the identification scan, suspicious regions or ROIs are identified. The ROI can be identified based on, for example, location, size, image texture of the ROI from the initial scan, the image contrast of the ROI from the initial scan, the existence of a tumor (according to a computer analysis of ROI from first scan), the absence of a tumor. The computer analysis of a ROI includes known computer-aided detection or computer-aided diagnosis techniques known to those skilled in the art.

Based on information found in the identification scan, the sequences for the subsequent scans are determined and programmed into the MRI system 100. The MRI system 100 executes the programmed scans, seamlessly switching from one set of scan parameters to the next.

In this embodiment, the suspicious regions are scanned with a HiSS scan (block 605) with high spectral and spatial resolution. This scan provides images prior to contrast injection containing valuable clinical information about the ROI. Properties of a high spectral and spatial scan can include, for example:
Spectral: <15 Hz
Spatial: 0.5 mm×0.5 mm×2.0 mm voxel
# of Slices: 32 saggital
Time: about 8 minutes with a SENSE acceleration factor of 3

Next a mask scan (block 610) is performed. The images generated by this scan can provide a basis to identify the impact of the contrast agent on images generated following administration of the contrast agent. Properties of a mask scan can include, for example:
Spectral: 60 Hz
Spatial: 1.5 mm×1.5 mm×4.0 mm voxel
# of Slices: 160 saggital
Repetition: 4 times

Next, a plurality of slices (for example, eight slices) of the ROI are chosen for scanning during contrast agent uptake (block 615). These slices are scanned with high temporal and spatial resolution and low (but not zero) spectral resolution. This scan can run from about one minute before contrast injection until about two minutes after contrast injection. Properties of a contrast agent uptake scan can include, for example:
Spectral: 60 Hz
Spatial: 1.0 mm×1.0 mm×4.0 mm voxel
# of Slices: 8 saggital
Time: about 4 to 8 seconds per slice

At a predetermined time after contrast agent injection (for example, about two minutes), a plurality of sets of images (for example, about five sets of images) equivalent to the masking scan are scanned (block 620). These images are subtracted from the masking images to obtain images representing the effects of the contrast agent. Properties of a high spectral and spatial scan can include, for example:
Spectral: 60 Hz
Spatial: 1.5 mm×1.5 mm×4.0 mm voxel
# of Slices: 160 saggital
Repetition: 5 times

The suspicious regions are scanned post-contrast. The suspicious regions can be scanned with a HiSS scan (block 625) with high spectral and spatial resolution. This scan can provide images showing the impact of the contrast agent, and can include valuable clinical information on the ROI. Properties of a high spectral and spatial scan can include, for example:
Spectral: <15 Hz
Spatial: 0.5 mm×0.5 mm×2.0 mm voxel
# of Slices: 32 saggital
Time: about 8 minutes

From the combination of these scans, clinically useful information about the breast lesion, such as malignancy, can be identified.

In some embodiments, dynamic allocation protocols can be used in combination with standard clinical procedures to test the effectiveness of protocols. The following example illustrates a protocol that can be followed for a MRI guided biopsy of a breast lesion that can develop clinically useful dynamic allocation protocols.

As part of a normal clinical exam for an MRI-guided biopsy of a breast lesion, a set of T₂-weighted fast spin echo images are generated before contrast agent injection. Accurate determination of contrast agent concentration requires knowledge of the sensitivity profile of a breast coil. A calibration scan can be performed to determine the sensitivity of the local coil at each point in an imaged volume. An EPSI scan over a large volume around a lesion and reference tissues is acquired at very low spectral resolution and low spatial resolution with signal detection by a body coil, and is repeated using the breast coil. The body coil can be assumed to have a homogenous RF field/pulse angle over the sensitive volume of the breast coil. Therefore, the ratio of the signal from the breast coil to the signal from the body coil yields a sensitivity map. In addition, the spectroscopic information provides information on phase and amplitude of signal in each coil element from each point in the sample.

T₁-weighted gradient echo images at four different tip angles can be used for estimation of pre-contrast T₁. These images can be acquired from eight 4 mm thick slices through the region of a suspicious lesion. This allows accurate determination of contrast agent concentration as a function of time (total time about 2 minutes).

Next, unilateral T₁-weighted spoiled grass images are acquired before contrast agent injection (pre-contrast mask as part of the standard clinical exam).

Multi-slice spectral/spatial images are acquired from 25 capital slices through the region to be biopsied. With spectral resolution of 5 Hz, and spatial matrix size of 256×256, HiSS data from twenty-five 3 mm slices can be acquired in less than four minutes. Since suspicious lesions are detected with high sensitivity without contrast media injection using HiSS, these images can help to identify the position of the suspicious lesions so that slices for rapid scans during contrast media uptake can be correctly selected.

Sagittal T₁-weighted spectral/spatial images with high temporal resolution (about 10 seconds), high spatial resolution (about less than 1 mm) and modest spectral resolution (about 50 Hz) from eight slices in the region to be biopsied (selected based on multi-slice spectral/spatial imaging above) before and for about 80 seconds after contrast agent injection. Approximately eight images are acquired after contrast agent injection.

Unilateral T₁-weighted spoiled grass images, post-contrast agent injection, are acquired and used to unambiguously identify lesion position (part of the standard clinical exam—run time about 1 minute).

High-resolution spectral/spatial images are acquired post-contrast agent from about 8-10 slices through the region that is to be biopsied. With spectral resolution of 5 Hz, and spatial matrix size of 256×256, HiSS data can be acquired from eight 3 mm slices in less than 1.5 minutes with parallel imaging, sampling multiple k-space lines, and partial-Fourier imaging. Only eight slices are imaged at this point so that the biopsy procedure can begin before most of the contrast agent is washed out.

The standard clinical procedure is followed for the MRI-guided biopsy. The position of the lesion is already located on spoiled grass and spectral/spatial images. The conventional spoiled grass images are repeated during the biopsy procedure as needed to insure accurate placement of the needle. Additional contrast can be injected if necessary as part of standard clinical practice to facilitate localization.

Following the dynamically allocated imaging modified clinical procedure; determination can be made as to whether a contrast media dynamic, calculated from high temporal, spectral, and spatial resolution data, is a useful diagnostic marker. To determine the value of each individual marker, it can be compared to the value of each parameter of the ‘truth’ determined from biopsy results. A cutoff value can be calculated for each parameter to optimize sensitivity and specificity for that parameter. The following methods can be useful in determining the value of a diagnostic marker and, therefore, the value of the dynamic allocation protocol used.

Motion artifacts in images can be corrected using the 3-D information gathered from the multiple slices imaged, before and after contrast injection.

T₁, T₂*, and resonance frequency following contrast agent injection are measured for each voxel from spectral/spatial data, as described above, and changes in these parameters are measured. The initial T₁ in each voxel is determined from images acquired with four different TRs taking into account the pulse angle in each voxel. Then the change in T₁ following contrast agent injection is calculated from TR, the pulse angle (assuming homogeneous B₁ of the body coil), the sensitivity map of the breast coil, the change in signal intensity, and the initial T₁. Contrast agent concentration can be calculated from:

C(t)=Δ(1/T₁)*(1/R₁)

where:
C(t) is the contrast agent concentration as a function of time, and
R₁ is the longitudinal relaxivity of the contrast agent (e.g., Gadolinium-DTPA (˜4.7 mM⁻¹ sec⁻¹ at 1.5 T)).

First pass effects on T₁ are analyzed to determine a product of the perfusion (or ‘flow’) times the contrast media extraction fraction (Kₜᵣₐₙₛ): A two-compartment model of the tumor (the intravascular versus the extravascular space) can be used to describe the redistribution of contrast agent following bolus injection. This model can predict contrast agent concentration C(t) as a function of time (t):

$$\frac{d C ( t )}{d t} = \frac{F \cdot E}{VT} \cdot ( Ca ( t ) - \frac{1}{λ} C ( t ) ) ,$$

where:
F is perfusion rate,
E is the fraction of contrast agent molecules extracted from capillaries during the mean transit time,
VT is volume accessible to water,
Ca(t) is contrast agent concentration in local arteries as a function of time, and
λ is the fraction of the volume VT accessible to the contrast agent.

Ca(t) is estimated from C(t) in a reference tissue near the lesion (e.g., chest wall muscle, or auxiliary muscle) for which ‘F’, ‘E’, ‘VT’ and ‘λ’ are known. A double reference tissue method which uses data from two different reference tissues to provide a more accurate estimate of Ca(t) can be used. Then the Ca(t) is used to obtain physiologic parameters for tumor voxels; F·E/VT and λ are varied using a recursive form of the second equation until a best fit to the data is obtained.

A radiologist can manually outline the lesion and the average value of Kₜᵣₐₙₛ(or F*E) in the lesion can be calculated. In addition, the average F*E in thel 5% of the voxels with largest F*E and the average value of λ in this same group of voxels is calculated (F*E_{MAX} and λ_{MAX}). The rationale for this is that the strongest indicators of malignancy are considered to be small regions with dense vasculature and strong angiogenic activity. The sensitivity and specificity of these parameters as markers for certain cancers can be evaluated using a biopsy as the standard. In addition, F*E can be calculated on a voxel-by-voxel basis to produce a parametric F*E image and the morphology of the lesion in this parametric image can be evaluated. Then the spiculation and internal heterogeneity is evaluated. The sensitivity and specificity of each of the quantitative and morphologic ratings can then be determined based on the biopsy result.

Next, areas under the curve (“AUC”) images are calculated. To do this, the time of arrival (tₐ) of the bolus in each pixel must be calculated. This is taken to be the time at which image intensity increases 2 root-mean-square noise units following contrast media injection. The contrast media concentration can be integrated beginning at tₐ and continuing for 30 seconds. The average value of AUC30 in the lesion and the average value in the 15% of lesion voxels with the largest AUC30 are determined (AUC30_{MAX}). In addition, parametric images of the AUC30 in each voxel is calculated and Radiologists can evaluate degree of spiculation, linearity (degree to which the lesion is linear in shape), and edge sharpness for each morphologic parameter. The sensitivity and specificity of these parameters are calculated based on comparison with biopsy results. High AUC30_{MAX} can indicate high grade cancers, while lower values can indicate low grade cancers or benign lesions.

Morphologic parameters in images derived from spectral/spatial data can be measured to determine whether these parameters are useful diagnostic markers. A large number of images can be generated from the water and fat spectra produced as described above. Focus can be placed on images with intensity proportional to water signal peak height, T₂*, and/or peak resonance frequency acquired pre- and post-contrast agent injection.

In some embodiments, HiSS images before contrast media uptake, difference images calculated from high temporal and spatial resolution and modest spectral resolution acquired before, and during the first 10-20 seconds after bolus arrival (post CA-pre CA), and difference images calculated from spectral/spatial data acquired at three minutes after contrast media injection can be evaluated to determine if they identify useful diagnostic markers. Evaluation of the morphology of the lesion in these images can use the following parameters: a) lesion speculation; b) edge sharpness; c) texture; d) inhomogeneous enhancement following contrast injection; e) rim enhancement; f) distension/deformation of ducts. The results of the evaluations for each parameter can be compared with the biopsy result to determine the sensitivity and specificity of each feature for diagnosis of cancer.

In addition to measurements of sensitivity and specificity for each parameter extracted from dynamically allocated MRI bandwidth datasets, water peak height images calculated from HiSS data can be directly compared to the conventional images that are also acquired as part of the protocol detailed above. This includes comparisons of signal-to-noise ratio and contrast-to-noise ratio for selected anatomic features (e.g., for distinct tumor regions and features of the parenchyma), efficiency of fat suppression and sensitivity to small amounts of water in predominantly fat voxels, and sharpness of edges based on the local intensity gradient. Advantages of images obtained through dynamically allocated MRI bandwidth images over conventional images can then be determined.

Embodiments of dynamic allocated MRI can be used with any MRI imaging method including diffusion weighted, T₁-weighted, T₂*-weighted, arterial spin labeling, and others.

In addition to contrast agent uptake and washout, embodiments of dynamically allocated MRI have application in cardiac imaging, respiratory gated images, arterial spin labeling, and magnetization transfer, brain function mapping, as well as other kinetically impacted imaging.

The embodiments described above and illustrated in the figures are presented by way of example only and are not intended as a limitation upon the concepts and principles of the present invention. As such, it will be appreciated by one having ordinary skill in the art that various changes are possible. For example, various aspects of the present invention are described above with reference to breast imaging in which contrast agents are employed during an MRI procedure. It should be noted that such imaging is only presented by way of example, and is not intended to be limiting regarding the scope or application of the present invention (e.g., bandwidth allocation only for certain areas of the body, certain types of ROIs, and contrast agent-enhanced MRI). The present invention finds application for MRI of a large number of different body areas, ROIs, and even MRI imaging not employing contrast agent.

As another example, the bandwidth allocation features described herein are not limited to any particular order or sequence of changes during an MRI procedure. For example, although it may be desirable to increase the temporal bandwidth allocation during an early stage of an MRI procedure (such as immediately upon and for a period of time after contrast agent introduction), and to later increase spectral and/or spatial bandwidth allocation at the expense of temporal bandwidth, other bandwidth allocation processes are possible. In some embodiments, temporal, spectral, and/or spatial bandwidths can be increased or decreased at any point between the beginning and end of an MRI procedure, and can be increased or decreased at multiple times during the MRI procedure. For example, modification of the bandwidth allocation can occur during the acquisition of a single image of the patient. Also, any one or two of the temporal, spectral, and spatial bandwidths can be increased or decreased at any point during an MRI procedure at the expense or benefit of either or both of the other bandwidths, respectively.

Thus, the invention provides, among other things, a method for dynamically allocating MRI bandwidth to enable the acquisition of images with spatial, spectral, and temporal resolutions that provide an improved degree of potential information based on morphological changes taking place in a ROI. Various features and advantages of the invention are set forth in the following claims.

1. A method of generating magnetic resonance images of a patient, the method comprising:
allocating a bandwidth for temporal resolution, spatial resolution, and spectral resolution;
acquiring images of the patient; and
semi-automatically modifying the bandwidth allocation of at least one of the temporal resolution, spatial resolution, and spectral resolution at the expense of at least one of the other two resolutions.

2. (canceled)

3. The method of claim 1, further comprising injecting the patient with a contrast agent, and wherein the act of acquiring images of the patient using the first bandwidth occurs during a time period wherein a contrast of an image of a region of interest of the patient has a relatively high variability.

4. The method of claim 1, further comprising injecting the patient with a contrast agent, and wherein the act of acquiring images of the patient using the second bandwidth occurs during a time period wherein a contrast of an image of a region of interest of the patient has a relatively low variability.

5. (canceled)

6. The method of claim 1, further comprising identifying a diagnostic marker based on one of the first set of images, the second set of images, and a difference between the first set of images and the second set of images.

7-12. (canceled)

13. The method of claim 1 further comprising:
injecting the patient with a contrast agent;
implementing a first imaging protocol including high temporal resolution and high spatial resolution;
continuing the first imaging protocol for a predetermined time period; and
semi-automatically implementing a second imaging protocol following the predetermined time period, the second imaging protocol including high spatial resolution and high spectral resolution.

14-17. (canceled)

18. The method of claim 13, further comprising semi-automatically implementing at least one additional imaging protocol following the second imaging protocol.

19. The method of claim 13, wherein the predetermined time period is substantially equal to a time period from when the contrast agent is injected into the patient to when the contrast agent begins to washout from a region of interest in the patient.

20. A method of generating magnetic resonance images of a patient, the method comprising:
defining a first imaging protocol having a bandwidth including a relatively high temporal resolution and a relatively high spatial resolution;
defining a second imaging protocol having a bandwidth including a relatively high spatial resolution and a relatively high spectral resolution;
injecting the patient with a contrast agent;
implementing the first imaging protocol;
detecting a triggering event; and
implementing the second imaging protocol following detection of the triggering event.

21. The method of claim 20, wherein the second imaging protocol is implemented semi-automatically.

22. The method of claim 20, further comprising defining at least one additional imaging protocol and implementing the at least one additional imaging protocol following detection of additional triggering events.

23. The method of claim 20, wherein the triggering event is a conclusion of a time period.

24. The method of claim 20, wherein the triggering event is a beginning of a washout of contrast agent from a region of interest.

25. The method of claim 20, wherein the first and second imaging protocols include one or more imaging techniques.

26. The method of claim 20, wherein the images depict changes in morphology of a region of interest in the patient.

27-29. (canceled)

30. A magnetic resonance imaging system comprising:
a housing including means for acquiring images of a patient; and
a computer program embodied by a computer readable medium capable of being executed by a computer, the computer program including
a setup module operable to allocate a bandwidth for temporal resolution, spatial resolution, and spectral resolution, and
a scanning module operable to semi-automatically modify the bandwidth allocation of at least one of the temporal resolution, the spatial resolution, and the spectral resolution at the expense of at least one of the other two resolutions.

31. The system of claim 30 wherein the setup module is further configured to create one or more imaging protocols.

32. The system of claim 31 further comprising a pulse sequence module configured to create a set of pulse sequences for gradient coils and radio-frequency transmitter coil of the system and a scanning module configured to execute the set of pulse sequences.

33. The system of claim 32, wherein the series of pulse sequences are based on the one or more imaging protocols.

34. The system of claim 32, wherein the series of pulse sequences allocate a bandwidth of the system including a spectral resolution, a spatial resolution, and a temporal resolution.

35. The system of claim 32, wherein the pulse sequence module can create, and the scanning module can execute, a plurality of sets of pulse sequences for multiple imaging protocols.

36. The system of claim 32, wherein the scanning module can automatically execute the plurality of sets of pulse sequences consecutively.

37-43. (canceled)

44. A magnetic resonance imaging system comprising:
a computer program embodied by a computer readable medium, the computer program including
a scanning module operable to perform a first magnetic resonance imaging scan, and
a selection module operable to receive input from a user to select a region of interest, the scanning module operable to perform a second scan with an allocation of bandwidth to the temporal resolution, spectral resolution, and spatial resolution different from the first scan, and based on the selected region of interest.

45. The system of claim 44, wherein the allocation of bandwidth in the second scan uses relatively high spectral resolution for imaging at least a part of the region of interest.

46. The system of claim 44, wherein the first scan gathers no spectral information.

47. The method of claim 1, wherein semi-automatically modifying the bandwidth allocation of at least one of the temporal resolution, spatial resolution, and spectral resolution at the expense of at least one of the other two resolutions includes automatically modifying the bandwidth allocation of at least one of the temporal resolution, spatial resolution, and spectral resolution at the expense of at least one of the other two resolutions.

48. The system of claim 30, wherein the scanning module being operable to semi-automatically modify the bandwidth allocation of at least one of the temporal resolution, the spatial resolution, and the spectral resolution at the expense of at least one of the other two resolutions includes automatically modifying the bandwidth allocation of at least one of the temporal resolution, the spatial resolution, and the spectral resolution at the expense of at least one of the other two resolutions.
